SEQUENCE LISTING (14 sequences)

SEQ ID NO: 1 - DNA, 292 bp, Artificial Sequence
  Synthetic Pseudomonas saitens KCTC 13107BP
tagacctgactgcagagcgaattcgggcctgattcagggcaaaaatggccagaaaccatttttcgcgtaaaaaatattcattcgcgagtgctttttttccaagcccgtttaaccctcaagccccggaaaccggggcttcgagcatttcaggcagcagaccgaagctaacatcgcagcaaactgtggttcaggggggttccaaagcgcacatttcgactatgatagctcggtgtgcccagttggcctgagcagcacagtactactgaaaatataagtttcttggagatacacc

SEQ ID NO: 2 - DNA, 3576 bp, Artificial Sequence
  Synthetic pTSa-EX1 vector
gaattcagccagcaagacagcgatagagggtagttatccacgtgaaaccgctaatgccccgcaaagccttgattcacggggctttccggcccgctccaaaaactatccacgtgaaatcgctaatcagggtacgtgaaatcgctaatcggagtacgtgaaatcgctaataaggtcacgtgaaatcgctaatcaaaaaggcacgtgagaacgctaatagccctttcagatcaacagcttgcaaacacccctcgctccggcaagtagttacagcaagtagtatgttcaattagcttttcaattatgaatatatatatcaattattggtcgcccttggcttgtggacaatgcgctacgcgcaccggctccgcccgtggacaaccgcaagcggttgcccaccgtcgagcgccagcgcctttgcccacaacccggcggccggccgcaacagatcgttttataaatttttttttttgaaaaagaaaaagcccgaaaggcggcaacctctcgggcttctggatttccgatcacctgtaagtcggacgttccgatcacctgtaacgatgcgtccggcgtagaggatccggagcttatcgactgcacggtgcaccaatgcttctggcgtcaggcagccatcggaagctgtggtatggctgtgcaggtcgtaaatcactgcataattcgtgtcgctcaaggcgcactcccgttctggataatgttttttgcgccgacatcataacggttctggcaaatattctgaaatgagctgttgacaattaatcatcggctcgtataatgtgtggaattgtgagcggataacaatttcacacagggacgagctattgattgggtaccgagctcgaattcgtacccggggatcctctagagtcgacctgcaggcatgcaagcttggctgttttggcggatgagagaagattttcagcctgatacagattaaatcagaacgcagaagcggtctgataaaacagaatttgcctggcggcagtagcgcggtggtcccacctgaccccatgccgaactcagaagtgaaacgccgtagcgccgatggtagtgtggggtctccccatgcgagagtagggaactgccaggcatcaaataaaacgaaaggctcagtcgaaagactgggcctttcgttttatctgttgtttgtcggtgaacgctctcctgagtaggacaaatccgccgggagcggatttgaacgttgcgaagcaacggcccggagggtggcgggcaggacgcccgccataaactgccaggcatcaaattaagcagaaggccatcctgacggatggcctttttgcaagaacatgtgagcacttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagcatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaattctcatgtttgacagcttatcatcgataagctttaatgcggtagtttatcacagttaaattgctaacgcagtcaggcaccgtgtatgaaatctaacaatgcgctcatcgtcatcctcggcaccgtcaccctggatgctgtaggcataggcttggttatgccggtactgccgggcctcttgcgggatatcgtccattccgacagcatcgccagtcactatggcgtgctgctagcgctatatgcgttgatgcaatttctatgcgcacccgttctcggagcactgtccgaccgctttggccgccgcccagtcctgctcgcttcgctacttggagccactatcgactacgcgatcatggcgaccacacccgtcctgtggatcctctacgccggacgcatcgtggccggcatcaccggcgccacaggtgcggttgctggcgcctatatcgccgacatcaccgatggggaagatcgggctcgccacttcgggctcatgagcgcttgtttcggcgtgggtatggtggcaggccccgtggccgggggactgttgggcgccatctccttgcatgcaccattccttgcggcggcggtgctcaacggcctcaacctactactgggctgcttcctaatgcaggagtcgcataagggagagcgtcgaccgatgcccttgagagccttcaacccagtcagctccttccggtgggcgcggggcatgactatcgtcgccgcacttatgactgtcttctttatcatgcaactcgtaggacaggtgccggcagcgctctgggtcattttcggcgaggaccgctttcgctggagcgcgacgatgatcggcctgtcgcttgcggtattcggaatcttgcacgccctcgctcaagccttcgtcactggtcccgccaccaaacgtttcggcgagaagcaggccattatcgccggcatggcggccgacgcgctgggctacgtcttgctggcgttcgcgacgcgaggctggatggccttccccattatgattcttctcgcttccggcggcatcgggatgcccgcgttgcaggccatgctgtccaggcaggtagatgacgaccatcagggacagcttcaaggatcgctcgcggctcttaccagcctaacttcgatcactggaccgctgatcgtcacggcgatttatgccgcctcggcgagcacatggaacgggttggcatggattgtaggcgccgccctataccttgtctgcctccccgcgttgcgtcgcggtgcatggagccgggccacctcgacctgaatggaagccggcggcacctcgctaacggattcaccactccaagaattggagccaatttttaaggcagttattggtgcccttaaacgcctggttgctacgcctgaataagtgataataagcggatgaatggcagaaattc

SEQ ID NO: 3 - DNA, 26 bp, Artificial Sequence
  Synthetic primer
atggagaaaaaaatcactggatatac

SEQ ID NO: 4 - DNA, 20 bp, Artificial Sequence
  Synthetic primer
tcacaggtatttattcggcg

SEQ ID NO: 5 - DNA, 32 bp, Artificial Sequence
  Synthetic primer
aataaatacctgtgatagacctgactgcagag

SEQ ID NO: 6 - DNA, 45 bp, Artificial Sequence
  Synthetic primer
gatttttttctccatggtgtatctccaagaaacttatattttcag

SEQ ID NO: 7 - DNA, 16 bp, Artificial Sequence
  Synthetic primer
tcaaatgcctgaggcc

SEQ ID NO: 8 - DNA, 17 bp, Artificial Sequence
  Synthetic primer
aaagggcctcgtgatac

SEQ ID NO: 9 - DNA, 32 bp, Artificial Sequence
  Synthetic primer
atcacgaggccctttggatccggagcttatcg

SEQ ID NO: 10 - DNA, 38 bp, Artificial Sequence
  Synthetic primer
gcctcaggcatttgaagatctaaaaggccatccgtcag

SEQ ID NO: 11 - protein, 236 aa, Artificial Sequence
  Synthetic Bacillus bombysepticus SF3
MKYKFILFDVDDTLLDFPETERHALHNAFVQFGMPTGYNDYLASYKEISNGLWRDLENKMITLSELAVDRFRQLFALHNIKVDAQQFSDVYLENLGKEVHLIEGAVQLCEDLQDCKLGIITNGYTKVQQSRIGNSPVCNFFDHIIISEEVGHQKPAREIFDYAFEKFGITDKSSVLMVGDSLSSDMRGGEDYGIDTCWYNPSLKENRTDVKPSYEVESLLQILEIVEVTKEKVASF

SEQ ID NO: 12 - DNA, 711 bp, Artificial Sequence
  Synthetic Bacillus bombysepticus SF3
atgaaatacaaatttatattattcgacgtagacgatacattattagatttccctgaaacggaaagacacgcattacataatgcgtttgtacagttcgggatgcctacagggtataatgattatcttgcaagttataaagagattagtaatggattatggagagatttagaaaataaaatgattacgctaagtgaattagcggtagatcgatttagacaattatttgcccttcataatataaaagtagatgcgcagcaatttagcgatgtatatcttgaaaacttagggaaagaagtacatcttatagaaggtgcagtgcaattatgtgaggatctacaagattgcaagttaggtattattacgaatggatatacgaaggtgcaacaatcgagaattggaaattcgcctgtatgtaatttctttgatcatattattatttcagaagaggttggtcatcaaaaaccagcacgtgagatttttgattatgcgtttgaaaagtttgggattacagataaatcaagtgtattaatggttggagattcgctttcttctgatatgagaggcggagaagattacggcattgatacgtgttggtataatccgagtttgaaagaaaataggacagatgttaagccgtcttatgaagtggagagtctgctacaaattttagaaattgtagaagtgactaaagaaaaagtagcttcattttaa

SEQ ID NO: 13 - DNA, 19 bp, Artificial Sequence
  Synthetic primer
attcacattcttgcccgcc

SEQ ID NO: 14 - DNA, 18 bp, Artificial Sequence
  Synthetic primer
tcaccgtaacacgccaca

(19) United States
(12) Patent Application Publication
(10) Pub. No.: US 2020/0024598 A1
(43) Pub. Date: Jan. 23, 2020

(54) ISOLATED POLYNUCLEOTIDE COMPRISING PROMOTER REGION, HOST CELL COMPRISING THE SAME, AND METHOD OF EXPRESSING TARGET GENE IN THE HOST CELL

(71) Applicant: Samsung Electronics Co., Ltd., Suwon-si (KR)

(72) Inventors: Soonchun Chung, Seoul (KR); Jinha Kim, Hwaseong-si (KR); Joonsong Park, Seoul (KR); Seunghee Cho, Seoul (KR)

(21) Appl. No.: 16/370,381

(22) Filed: Mar. 29, 2019

(30) Foreign Application Priority Data
Jul. 23, 2018 (KR) 10-2018-0085529

(51) Int. Cl. C12N 15/113; C12N 15/70; C12N 15/74; C12P 21/02; C12N 1/20; C12R 1/07; C12R 1/38; C12R 1/19

(52) U.S. Cl. CPC C12N 15/113; C12N 15/70; C12N 15/74; C12R 1/19; C12N 1/20; C12R 1/07; C12R 1/38; C12P 21/02

(57) ABSTRACT

Provided are an isolated polynucleotide, a host cell including the isolated polynucleotide, and a method of expressing a target gene in the host cell, wherein the isolated polynucleotide includes a promoter region derived from a bacterium of the genus Pseudomonas.

### CROSS-REFERENCE TO RELATED APPLICATION

This application claims the benefit of Korean Patent Application No. 10-2018-0085529, filed on Jul. 23, 2018, in the Korean Intellectual Property Office, the entire disclosure of which is hereby incorporated by reference.

### INCORPORATION-BY-REFERENCE OF MATERIAL SUBMITTED ELECTRONICALLY

Incorporated by reference in its entirety herein is a computer-readable nucleotide/amino acid sequence listing submitted concurrently herewith and identified as follows: One 10,627 Byte ASCII (Text) file named “741177_ST25.TXT,” created on Mar. 28, 2019.

### BACKGROUND

### 1. Field

The present disclosure relates to an isolated polynucleotide including a promoter region derived from a bacterium of the genus Pseudomonas, a host cell including the isolated polynucleotide, and a method of expressing a target gene in the host cell.

### 2. Description of the Related Art

An expression promoter is required for expression of a target gene in a microorganism. The promoter is a region where RNA polymerase binds to initiate transcription of DNA into mRNA. The binding of RNA polymerase and, in some cases, other transcription factors to the promoter and the strength of said binding depend at least in part upon the nucleotide sequence and length of the promoter. In other words, the promoter determines expression strength and conditions of the gene.

Among microorganisms, bacteria are often used to produce useful products. Bacteria include Gram-negative and Gram-positive bacteria. Gram-negative bacteria include Pseudomonas and Escherichia. Gram-positive bacteria include Bacillus.

Accordingly, there is a need for a promoter capable of expressing a target gene at a desired strength in bacteria.

### SUMMARY

Provided is an isolated polynucleotide comprising a promoter region having a nucleotide sequence of SEQ ID NO: 1.

Also provided is a host cell comprising the polynucleotide.

Further provided is a method of expressing a target gene in the host cell using the polynucleotide.

### BRIEF DESCRIPTION OF THE DRAWINGS

These and/or other aspects will become apparent and more readily appreciated from the following description of the embodiments, taken in conjunction with the accompanying drawings in which:

FIG. 1 illustrates a map of a pB-P4381 vector;

FIG. 2 illustrates a map of a pSF-EX1 vector;

FIG. 3 illustrates a map of a pBBR-122 vector;

FIG. 4 shows cat mRNA levels of Pcat, Ptac, and P4381, as determined by RT-PCR; and

FIG. 5 shows results of SDS-PAGE analysis of lysates of Pcat-cm^{R}, Ptac-cm^{R}, and P4381-cm^{R} cells.

### DETAILED DESCRIPTION

Reference will now be made in detail to particular embodiments, examples of which are illustrated in the accompanying drawings, wherein like reference numerals refer to like elements throughout. In this regard, the present embodiments may have different forms and should not be construed as being limited to the descriptions set forth herein. Accordingly, the embodiments are merely described below, by referring to the figures, to explain aspects of the present description.

An aspect of the present disclosure provides an isolated polynucleotide comprising a promoter region comprising, consisting essentially of, or consisting of a nucleotide sequence of SEQ ID NO: 1.

The term “promoter” as used herein refers to a DNA region to which an RNA polymerase binds so as to initiate transcription of a gene operably linked to the promoter. The nucleotide sequence of the promoter may be genetically modified (e.g., through nucleotide substitution, addition, deletion, etc.) to the extent the modified promoter shares the same or similar function as an unmodified promoter of the same nucleotide sequence. Thus, embodiments of the present disclosure include a promoter having a sequence identity of, for example, 70% or higher, 80% or higher, 90% or higher, 95% or higher, or 97% or higher, or 99% or higher to the nucleotide sequence of SEQ ID NO: 1. In some embodiments, the promoter may consist of the nucleotide sequence of SEQ ID NO: 1.

The isolated polynucleotide may be an expression cassette or a vector. In one embodiment of the present disclosure, the polynucleotide (e.g., expression cassette or vector) may comprise a target gene operably linked to a promoter region. For example, the target gene may be operably linked to the promoter and positioned downstream of the promoter region. The term “operably linked” as used herein means that a gene to be expressed (e.g., “the target gene”) is functionally linked to its control (regulatory) sequences. Thus, a target gene operably linked to a promoter is expressed under the control of the promoter. The vector may further include any additional elements typically included in expression vectors, such as a replication origin, a promoter control site, a ribosome binding site, a transcription termination site, a selection marker, or a combination thereof, as well as the promoter or variant thereof and the target gene. The target gene may be a gene that is derived from an origin different from that of the promoter region (i.e., heterologous to the promoter). In a further embodiment, the gene is derived from the same source as the promoter region but is a gene that differs from the gene originally or naturally linked to the promoter region. In other words, the promoter region may be operably linked to a gene other than a naturally occurring gene to which the promoter region is originally linked, and thus, expression thereof is promoted.

The term “vector” refers to a nucleic acid molecule or construct capable of transferring another nucleic acid linked thereto (e.g., a gene or expression “cassette”) to a host cell. The vector may be, for example, a plasmid (e.g., a plasmid expression vector), a vector derived from a virus (e.g., a viral expression vector), or any other type of vector known in the art. The plasmids refer to circular, double-stranded DNA rings to which additional DNAs may be linked. A viral vector may be, for example, a replication-defective retrovirus, an adenovirus, an adeno-associated virus, a lentivirus, or any of other various types of viruses known in the art for gene delivery. The promoter can be heterologous to the vector. In the viral vector, additional DNA may be linked to the viral genome.

Certain vectors are capable of autonomous replication in a host cell into which they are introduced (e.g., bacterial vectors having a bacterial origin of replication). Other vectors are integrated into the genome of a host cell upon introduction into the host cell, and are thereby replicated along with the host genome. Moreover, certain vectors may direct expression of genes to which they are operatively linked. Such vectors are referred to herein as “expression vectors”. In general, expression vectors of utility in recombinant DNA techniques are often in the form of plasmids. The vector used herein may include, for example, a plasmid expression vector, a viral expression vector, and a viral vector capable of performing a function equivalent thereto.

The vectors of the present disclosure, such as expression vectors, can include one or more regulatory sequences, selected on the basis of the host cells to be used for expression, and are operatively linked to the nucleic acid sequence to be expressed. “Operatively linked” means that the nucleotide sequence of interest is linked to the regulatory sequence(s) in a manner which allows for expression of the nucleotide sequence in the host cell. The term “regulatory sequence” is intended to include promoters, enhancers, and other expression control elements (e.g., polyadenylation signals). Regulatory sequences include sequences which direct constitutive expression of a target nucleic acid in many types of host cells and sequences that direct expression of the target nucleic acid only in particular host cells (e.g., tissue-specific regulatory sequences). It will be appreciated by those skilled in the art that design of the expression vector may depend on factors such as selection of the host cell to be transformed, an expression level of a protein desired, or the like. The expression vector of the present disclosure may be introduced into the host cell to express the protein.

The plasmid may be a bacterial cloning vector. These cloning vectors may include a site that allows DNA to be inserted, for example, a multiple cloning site or a polylinker having several commonly used restriction sites to which DNA fragments may be ligated. After the gene of interest is inserted, the plasmids are introduced into bacteria by transformation. These plasmids may comprise a selectable marker, usually, an antibiotic resistance gene, which confers on the bacteria an ability to survive and proliferate in a selective growth medium containing the particular antibiotics. The cells after transformation may then be exposed to the selective media, and thus, only cells containing the plasmid may survive. In this way, the antibiotics act as a filter to select only the bacteria containing the plasmid DNA. The vector may also comprise other marker genes or reporter genes to facilitate selection of plasmid with the cloned insert. Thereafter, bacteria containing the plasmid may be grown in large amounts, harvested, and then isolated using various methods of plasmid preparation. In some embodiments, the plasmid cloning vector may be used to clone DNA fragments of about 15 kbp or shorter. The vector may be a commercially available vector, for example, a pBBR112, pBR322, pUC, TOPO cloning vector, etc.

In one aspect of the present disclosure, the target gene may encode a target protein. In certain embodiments, the target gene may encode a protein of the genus Pseudomonas, for example, Pseudomonas saitens. The gene encoding the target product (e.g., target protein) may be a gene encoding a Gene_0757 protein comprising SEQ ID NO: 11. In another embodiment, the gene encoding the target product may be a gene comprising SEQ ID NO: 12 that encodes a Gene_0757 protein. The isolated polynucleotide may be recombinant, artificial synthetic, or artificial semisynthetic.

Under aerobic conditions, the isolated polynucleotide may increase expression (e.g., mRNA level) of the target gene operably linked to the promoter region by about 32 times or higher, as compared with that of the gene operably linked to a tac promoter.

Another aspect of the disclosure provides a host cell comprising the polynucleotide comprising the promoter region that comprises the nucleotide sequence of SEQ ID NO: 1. The polynucleotide may be introduced into the cell from the outside (e.g., introduced exogenously).

The host cell may be a Gram-negative or Gram-positive bacterium. The Gram-negative bacterium may be, for example, a bacterium of the genus Pseudomonas or Escherichia. The Gram-positive bacterium may be, for example, a bacterium of the genus Bacillus. In certain embodiments, the host cell may be Pseudomonas saitens, Bacillus bombysepticus, or Escherichia coli.

In the host cell, the polynucleotide may be in a vector. In the vector, the target gene may be operably linked to the promoter region. The target gene may encode a target protein. The target gene may encode a protein of the genus Pseudomonas, for example, a protein of Pseudomonas saitens. The gene encoding the target product may be a gene encoding a Gene_0757 protein comprising SEQ ID NO: 11. The gene encoding the target product may be a gene comprising SEQ ID NO: 12 that encodes a Gene_0757 protein. The isolated polynucleotide may be recombinant, artificial synthetic, or artificial semisynthetic.

The vector may be introduced into the host cell, for example, to clone the target gene or to express the target gene. The introduction of the vector may be performed by applying appropriate standard techniques known in the art, depending on the host cell, for example, by electroporation, heat-shock, calcium phosphate (CaPO₄) precipitation, calcium chloride (CaCl₂)) precipitation, microinjection, a polyethylene glycol (PEG) method, a DEAE-dextran method, a cationic liposome method, a lithium acetate-DMSO method, or a combination thereof.

The vector introduced into the host cell may comprise a gene involved in production of the target protein that is operably linked downstream of the promoter or a variant thereof. The vector may further comprise a replication origin, a promoter control site, a ribosome binding site, a transcription termination site, a selection marker, or a combination thereof, as well as the promoter or variant thereof and the target gene. The host cell may express the gene operably linked to the promoter or variant thereof, for example, under anaerobic conditions. For example, the gene may be highly expressed under aerobic conditions and may also maintain a relatively high level of gene expression under anaerobic conditions. Under aerobic conditions, an expression level (e.g., mRNA level) of the gene may be 32 times or more (e.g., 50 times or more, or even 100 times or more) than that of the gene operably linked to the tac promoter.

The host cell may be a recombinant strain. In some embodiments, the promoter, the target gene, or both, are heterologous to the host cell.

Still another aspect of the present disclosure provides a method of expressing the target gene, the method comprising culturing the host cell comprising the polynucleotide comprising the promoter region that comprises the nucleotide sequence of SEQ ID NO: 1, wherein the target gene is operably linked to the promoter region to express the target gene.

For example, the method includes culturing the host cell comprising the polynucleotide comprising the promoter region that comprises the nucleotide sequence of SEQ ID NO: 1, wherein the target gene is operably linked to the promoter region to express the target gene. The host cell may be the same as the host cell described above.

In the method of the present disclosure, the host cell may be a Gram-negative or Gram-positive bacterium. The Gram-negative bacterium may be, for example, a bacterium of the genus Pseudomonas or Escherichia. The Gram-positive bacterium may be, for example, a bacterium of the genus Bacillus. The host cell may be Pseudomonas saitens, Bacillus bombysepticus, or Escherichia coli.

The target gene may encode a target protein. The target gene may encode a protein of the genus Pseudomonas, for example, a protein of Pseudomonas saitens. The gene encoding the target product may be a gene encoding a Gene_0757 protein comprising SEQ ID NO: 11. The gene encoding the target product may be a gene comprising SEQ ID NO: 12 that encodes a Gene_0757 protein. The isolated polynucleotide may be recombinant, artificial synthetic, or artificial semisynthetic. In the host cell, the polynucleotide may be a vector. In the polynucleotide or vector, the target gene may be operably linked to the promoter region.

The method may include, for example, a method of culturing the vector-introduced host cells to produce a final product in a biosynthetic pathway involving the protein encoded by the target gene. The target gene may be, for example, involved in production of a product selected from the group consisting of proteins, cellulose, L-amino acids, lactic acid, acetic acid, succinic acid, and combinations thereof. Therefore, the method may be used to produce the final product of the gene, for example, a product selected from the group consisting of proteins, cellulose, L-amino acids, lactic acid, acetic acid, succinic acid, and combinations thereof under aerobic and/or anaerobic conditions. In the method, the product may be, for example, produced in a large amount under aerobic conditions and also maintained in a relatively large amount under anaerobic conditions. The host cell may be, for example, Pseudomonas saitens KCTC 13107BP that is introduced with a vector, in which the gene involved in the production of the product is operably linked to the promoter having the nucleotide sequence of SEQ ID NO: 1 or a variant thereof.

The culturing of the host cell may be performed according to general methods known in the art. A medium used for the culturing may comprise a sugar source, for example: sugar and carbohydrate e.g., glucose, saccharose, lactose, fructose, maltose, starch, and cellulose; oil and fat, e.g., soybean oil, sunflower oil, castor oil, coconut oil, etc.; a fatty acid, e.g., palmitic acid, stearic acid, and linolenic acid; an alcohol, e.g., glycerol and ethanol; and an organic acid, e.g., acetic acid, individually or in a mixture. The medium may comprise a nitrogen source, for example: peptone, a yeast extract, a meat extract, a malt extract, corn steep liquor, soy meal and urea, or an inorganic compound, e.g., ammonium sulfate, ammonium chloride, ammonium phosphate, ammonium carbonate, or ammonium nitrate, individually or in a mixture. The medium may comprise a phosphorous source, for example, potassium dihydrogen phosphate, dipotassium hydrogen phosphate, or a corresponding sodium-containing salt thereof. The medium may comprise, for example, a metal salt, e.g., magnesium sulfate or iron sulfate, which is beneficial for growth. Also, during culturing, substances helpful for growth, such as amino acids and vitamins, or suitable precursors, may be added to the culture. Those components may be added to the culture in a manner known in the art, for example, in a batch or continuous manner during the culturing.

In some embodiments, the culturing may be performed under aerobic conditions.

According to an embodiment of the isolated polynucleotide of the present disclosure, gene transcription may be efficiently initiated in cells.

According to an embodiment of the host cell of the present disclosure, transcription of the target gene may be efficiently initiated.

According to an embodiment of the method of expressing the target gene of the present disclosure, the target gene may be efficiently expressed.

Hereinafter, the present disclosure will be described in more detail with reference to Examples. However, these Examples are for illustrative purposes only, and the scope of the present disclosure is not intended to be limited by these Examples.

### Example 1: Exploration and Identification of Promoter

In this Example, genes overexpressed in Pseudomonas saitens KCTC 13107BP microorganism (hereinafter, referred to as ‘SF1 strain’) were selected and promoters of the genes were isolated, respectively. Vectors, each vector including the promoter and a gene encoding a protein operably linked to the promoter, were prepared and introduced into SF1 strain to examine promoter strength.

The SF1 strain was deposited at the Korea Collection for Type Culture (KCTC) on Sep. 12, 2016 under accession number KCTC 13107BP.

(1) Exploration of Promoter

Pseudomonas saitens KCTC 13107BP was seeded in a serum bottle containing 10 ml of an LB medium per bottle, and subjected to static culture under aerobic conditions at 30° C. for 91 hours until OD₆₀₀=5.0. 10 g/L of tryptone, 5 g/L of yeast extract, and 10 g/L of NaCl were included in the LB medium.

Cells were isolated from the obtained culture, and a cell lysate was obtained. Genes showing high expression levels were identified by an RNA sequencing (RNA-seq) method. The RNA-seq method, also called whole transcriptome shotgun sequencing (WTSS), is a technique capable of examining the presence and amount of RNA in a biological sample at a given point by next-generation sequencing (NGS). Gene_4381 was selected from among the genes, and a promoter polynucleotide comprising SEQ ID NO: 1 was isolated from Gene 4381.

### (2) Construction of Vector

A pB-P4381 vector including the promoter obtained in section (1), above, was constructed as follows. A pBBR-122 vector (MoBiTec) was used as a template and 122-CmORF_F/122-Cmpro_R primers (SEQ ID NOS: 3 and 4) were used to amplify part of the vector. The promoter region selected in section (1) above was amplified using genomic DNA of the SF1 strain as a template and a set of P4381_F/P4381_R primers (SEQ ID NOS: 5 and 6). The promoter region was then cloned into the amplified vector backbone using an IN-FUSION® GD cloning kit (Takara).

A pSF-EX1 vector was constructed as follows. The pBBR-122 vector as a template and P1831/P1832 primers (SEQ ID NOS: 7 and 8) were used to amplify part of the vector. A tac promoter which was amplified using pTSa-EX1 (SEQ ID NO: 2) as a template and P1833/P1834 primers (SEQ ID NOS: 9 and 10) and an rrnB terminator region were cloned into the amplified vector backbone using the IN-FUSION GD cloning kit (Takara).

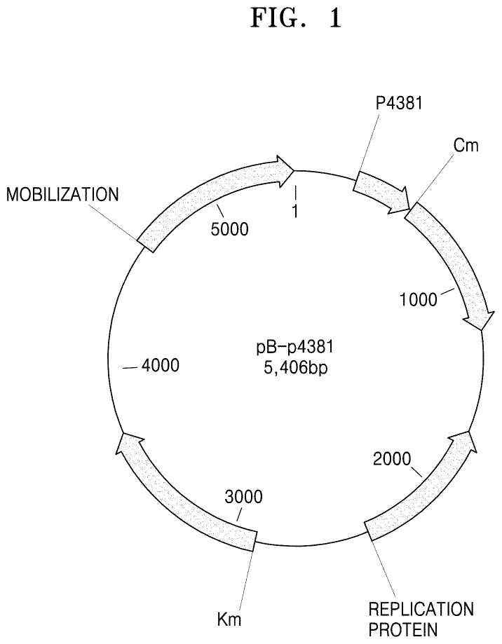

FIG. 1 illustrates a map of the pB-P4381 vector.

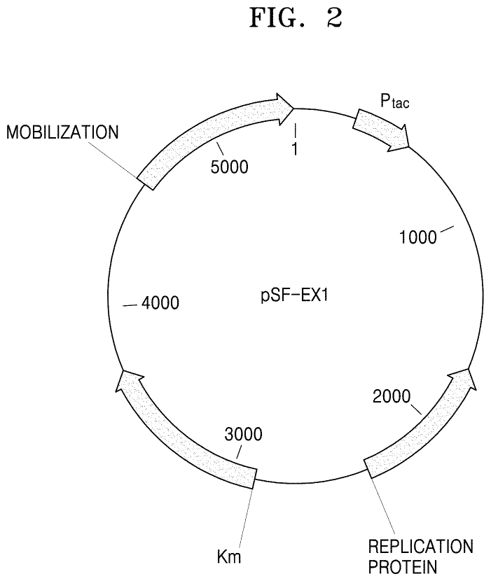

FIG. 2 illustrates a map of the pSF-EX1 vector.

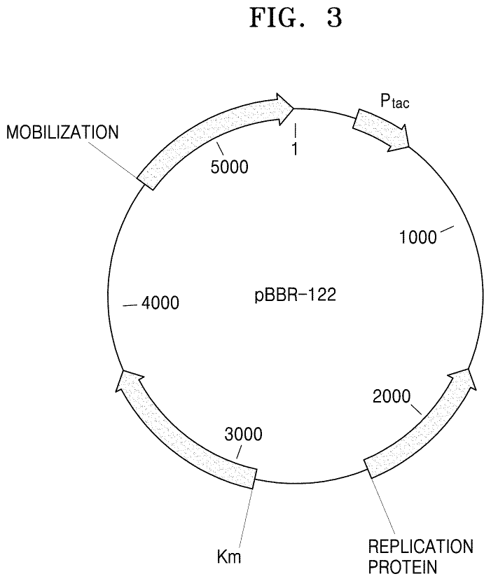

FIG. 3 illustrates a map of the pBBR-122 vector.

The pB-P4381, pSF-EX1, and pBBR-122 vectors were identical, except that the promoters linked to a cat gene encoding chloramphenicol (Cm) acetyltransferase were P4381, Ptac, and Pcat, respectively.

(3) Comparative Evaluation of Promoter Performances

A pB-P4381 vector comprising cm^{R} gene (cat) downstream of the promoter of highly expressed gene Gene_4381, which was selected based on RNA-seq data, was constructed as described in (2). This vector was then introduced into SF1 strain by electroporation. As control groups, a pSF-EX1 vector and a pBBR-122 vector were introduced into separate SF1 strains in the same manner as described above.

(3.1) MIC Test

Each of the recombinant SF1 strains was cultured in separate wells of a microplate containing LB medium containing predetermined chloramphenicol, and growth thereof was examined to confirm minimal inhibitory concentrations (MICs). Culturing was performed at 30° C. for 2 days in an LB medium supplemented with 400 μg/ml of chloramphenicol by ½ dilution at each concentration. Table 1 shows MIC of chloramphenicol of SF1 strains introduced with pB-P4381 vector, pSF-EX1 vector, and pBBR-122 vector, respectively.

TABLE 1

| Strain | MIC of Chloramphenicol (μg/ml) |
|---|---|
| Pcat-cm^{R} | >100 |
| Ptac-cm^{R} | >100 |
| P4381-cm^{R} | >200 |

As shown in Table 1, P4381 promoter showed MIC twice or higher than that of existing Pcat and Ptac, indicating that performance of P4381 is stronger than those of other promoters, resulting in high cat expression. In Table 1, Pcat-cm^{R}, Ptac-cm^{R}, and P4381-cm^{R} represent SF1 strains introduced with pB-P4381 vector, pSF-EX1 vector, and pBBR-122 vector, respectively.

(3.2) Examination of Cat Gene Transcription

Pcat-cm^{R}, Ptac-cm^{R}, and P4381-cm^{R} cells cultured in (3.1) were lysed to obtain cell lysates, which were then centrifuged to obtain supernatants, respectively. Each supernatant was subjected to RT-PCR using a set of primers of SEQ ID NOS: 13 and 14.

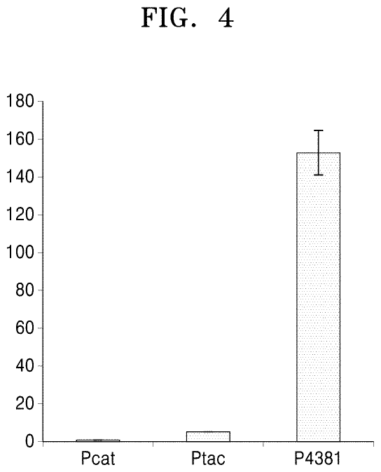

FIG. 4 shows cat mRNA levels, as determined by RT-PCR. As shown in FIG. 4, when P4381 promoter was used, the cat mRNA level was 158 times and 32 times higher than those of Pcat and Ptac, indicating that P4381 promoter has higher transcription initiation-promoting ability than Pcat and Ptac. In FIG. 4, the vertical axis represents a ratio when a cycle threshold (CT) value of Pcat promoter is regarded as 1, and Pcat, Ptac, and P4381 on the horizontal axis represent SF1 strains introduced with pB-P4381 vector, pSF-EX1 vector, and pBBR-122 vector, respectively.

(3.3) Examination of Cat Gene Transcription

Pcat-cm^{R}, Ptac-cm^{R}, and P4381-cm^{R} cells cultured in (3.1) were lysed to obtain cell lysates, from which soluble proteins were isolated using a BuGBusTER® Protein Extraction Reagent, respectively. The isolated proteins were subjected to SDS-PAGE to examine cat expression.

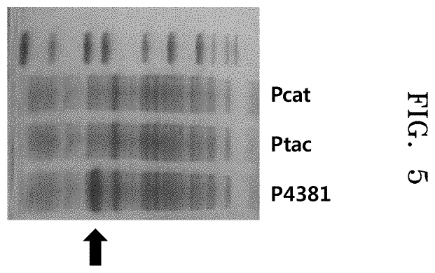

FIG. 5 shows the results of SDS-PAGE analysis of cell lysates of Pcat-cm^{R}, Ptac-cm^{R}, and P4381-cm^{R} cells. As shown in FIG. 5, when P4381 promoter was used, a cat protein expression level was significantly higher than those of Pcat and Ptac, indicating that P4381 promoter has higher transcription initiation-promoting ability than Pcat and Ptac. In FIG. 5, the arrow indicates the position of cat protein.

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and “at least one” and similar referents in the context of describing the invention (especially in the context of the following claims) are to be construed to cover both the singular and the plural, unless otherwise indicated herein or clearly contradicted by context. The use of the term “at least one” followed by a list of one or more items (for example, “at least one of A and B”) is to be construed to mean one item selected from the listed items (A or B) or any combination of two or more of the listed items (A and B), unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

What is claimed is:

1. An isolated polynucleotide comprising a promoter region comprising the nucleotide sequence of SEQ ID NO: 1.

2. The isolated polynucleotide of claim 1, wherein the isolated polynucleotide consists of the nucleotide sequence of SEQ ID NO: 1.

3. The isolated polynucleotide of claim 1, wherein the isolated polynucleotide is a vector.

4. The isolated polynucleotide of claim 1, wherein the isolated polynucleotide further comprises a target gene operably linked to the promoter region, wherein the target gene is heterologous to the promoter.

5. The isolated polynucleotide of claim 4, wherein the target gene encodes a target protein.

6. The isolated polynucleotide of claim 5, wherein the target gene encodes a protein comprising the amino acid sequence of SEQ ID NO: 11.

7. A host cell comprising a polynucleotide of claim 1.

8. The host cell of claim 7, wherein the polynucleotide is exogenous to the host cell.

9. The host cell of claim 7, wherein the host cell is a bacterial cell.

10. The host cell of claim 7, wherein the host cell is a Pseudomonas, Bacillus, or Escherichia cell.

11. The host cell of claim 10, wherein the host cell is Pseudomonas saitens, Bacillus bombysepticus, or Escherichia coli.

12. The host cell of claim 7, wherein the polynucleotide is a vector.

13. The host cell of claim 7, wherein the polynucleotide further comprises a target gene operably linked to the promoter region.

14. The host cell of claim 13, wherein the target gene encodes a target protein.

15. The host cell of claim 13, wherein the target gene encodes a protein comprising the amino acid sequence of SEQ ID NO: 11.

16. A method of expressing a target gene in a host cell, the method comprising culturing a host cell comprising a polynucleotide of claim 4, such that the target gene is expressed.

17. The method of claim 16, wherein the host cell is a bacterial cell.

18. The method of claim 16, wherein the target gene encodes a target protein.

19. The method of claim 16, wherein the culturing is performed under aerobic conditions.
